(19) United States
(12) Patent Application Publication
(10) Pub. No.: US 2024/0399373 A1
(43) Pub. Date: Dec. 5, 2024

(54) TEST SAMPLE TIMER TRAY

(71) Applicant: Assaya LLC, Roswell, GA (US)

(72) Inventor: Clas Sivertsen, Lilburn, GA (US)

(21) Appl. No.: 17/365,930

(22) Filed: Jul. 1, 2021

(51) Int. Cl. B01L 3/00; G06F 3/14; G06F 3/147

(52) U.S. Cl. CPC B01L 3/508; G06F 3/147; B01L 2200/04; B01L 2300/027; B01L 2300/043; B01L 2300/0609; B01L 2300/0663; G06F 3/1423

(57) ABSTRACT

A device including a processor and memory coupled to the processor. The device has a display and a switch coupled to the processor. The device has a cassette holder for holding a cassette associated with the display and the switch. When the processor receives an input indicating activation of the switch, a timer is activated for a predetermined timer length and time is displayed on the display.

### BACKGROUND

The present invention generally relates to sample trays and more specifically, to a test sample tray system and method.

Coronavirus Disease 2019 (“COVID-19”) is spreading throughout the country and the world caused by the spread of a novel coronavirus called SARS-COV-2. With the rapid spread of the disease, testing quickly, accurately, and efficiently is becoming more important. Testing may be performed using a lateral flow assay (“LFA”) strip present in an assay tube or cassette. The results of the test are determined after an incubation period following application of a test sample to a test pad of the LFA strip. The incubation period following application of the test sample to the sample pad of the LFA strip must be carefully timed to ensure that the proper time period has elapsed, but not too much time has elapsed, prior to examining the LFA strip to determine test results.

### SUMMARY

Embodiments of the present invention are directed to a device having a processor and memory coupled to the processor is provided. The device has a display and a switch coupled to the processor. The device has a cassette holder for holding a cassette associated with the display and the switch. When the processor receives an input indicating activation of the switch, a timer is activated for a predetermined timer length and time is displayed on the display.

Additional embodiments of the present invention are directed to a device having a processor and memory coupled to the processor. The device has a sensor coupled to the processor and a receptacle for holding a sample associated with the switch. When the processor receives an input indicating activation of the sensor, a timer is activated for a predetermined timer length.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts an orthogonal view of a timer tray with an open lid according to a first embodiment of the present invention;

FIG. 2 depicts an orthogonal view of the timer tray with the lid closed according to the first embodiment of the present invention;

FIG. 3 depicts a top view of the timer tray according to the first embodiment of the present invention;

FIG. 4 depicts an orthogonal view of the underside of the timer tray with the circuit board removed according to the first embodiment of the present invention;

FIG. 5 depicts an orthogonal view of a cross-section of the timer tray at the start switch below the interface panel according to the first embodiment of the present invention;

FIG. 6 depicts an orthogonal view of the hinge of the lid of the timer tray according to the first embodiment of the present invention;

FIG. 7 depicts an orthogonal view of the underside of the circuit board of the timer tray according to the first embodiment of the present invention;

FIG. 8 depicts a block diagram of the electronic circuitry of the timer tray according to the first or a second embodiment of the present invention;

FIG. 9 depicts a flow chart of the operation of the electronic circuitry of the timer tray according to the first embodiment of the present invention;

FIG. 10 depicts an orthogonal view of a timer tray in accordance with a second embodiment of the present invention;

FIG. 11 depicts the capacitance switch and multicolor LED placed at the bottom of each receptacle in accordance with the second embodiment of the present invention; and

FIG. 12 depicts a flow chart of the operation of the electronic circuitry of the timer tray according to the second embodiment of the present invention.

The diagrams depicted herein are illustrative. There can be many variations to the diagrams or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two-or three-digit reference numbers. With minor exceptions, the leftmost digit(s) of each reference number corresponds to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, as previously stated, SARS-COV-2, also often called SARS2 for short, is spreading rapidly around the country and around the world resulting in a large portion of the population being at risk of developing COVID-19. It is imperative to mass test, often repeatedly, the population for SARS-COV-2, and present testing systems are inadequate.

A polymerase chain reaction (“PCR”) test is currently the gold standard, as it is most sensitive. But because of the massive volumes of tests and due to the multitude of steps in collecting, storing, shipping, receiving, extracting, preparing, actually testing, collecting results, and distributing results, the actual result data have shown to be error prone due to the many possibilities of human error along the way. Because PCR tests are so sensitive there is also a high risk of cross contamination. Coupling that with the high increase in the volume of such tests and the shortage of skilled labor in laboratories, this further adds to the actual inaccuracy in the test data.

Rapid antigen and antibody tests and associated reading methods become crucial in improving the speed and quality of test data. These tests use LFA strips that are usually mounted within a cassette. During the remainder of this description, whenever the term “cassette” is used that term includes any of an assay tube, cassette, or card. The LFA strip provides indicators (stripes at various places along the LFA strip) that indicate the presence of analytes present in a sample. Once a test sample is placed on the sample pad of the LFA strip an incubation period must pass before the LFA strip test results should be read in order to determine positive or negative results. Presently, the cassettes are timed manually using a technician's watch or at best a stand-alone timer that may or may not be placed near the cassette. Visits to testing labs or testing sites show scores of cassettes awaiting completion of incubation with lab technicians often using sticky notes, again at best, to associate a timer with its respective cassette. Needless to say, incubation times are often missed with incubation incomplete or overtime before the LFA strip result is read. Frankly, at mass testing sites it can be chaos.

Embodiments of the present invention use a timer tray that has a plurality of timers associated with a respective plurality of cassette holders. When a cassette is placed into the timer tray that, for example, holds ten cassettes, the technician presses a switch by the cassette holder in which the cassette has been placed. A timer by the switch and the cassette then begins to count down from a preset incubation time and a hold light, for example, a yellow light, is turned on by the cassette. This denotes that the incubation period is in process. Following completion of the incubation time, a ready light, for example, a green light, is turned on and the hold light is turned off. A sound may also be emitted. The technician may then remove the cassette and press the switch indicating that the cassette has been removed. If the technician fails to remove the cassette prior to an expiration period for reading the LFA strip, an expiration light, for example, a red light, is lit and the ready light turned off.

In an alternative embodiment, the switches by the cassette holders are replaced with sensors below the cassette holders that sense the presence and absence of samples and initiate timing automatically, without the need for switch presses from the technician.

As can be seen from the above description, embodiments of the present invention significantly reduce, if not eliminate, incubation period timing errors by automating the timing process or at least tightly associating timers to their respective cassettes. This greatly enhances lab efficiency, but more importantly, produces a far greater likelihood of getting accurate test results through reduction of incubation timing errors.

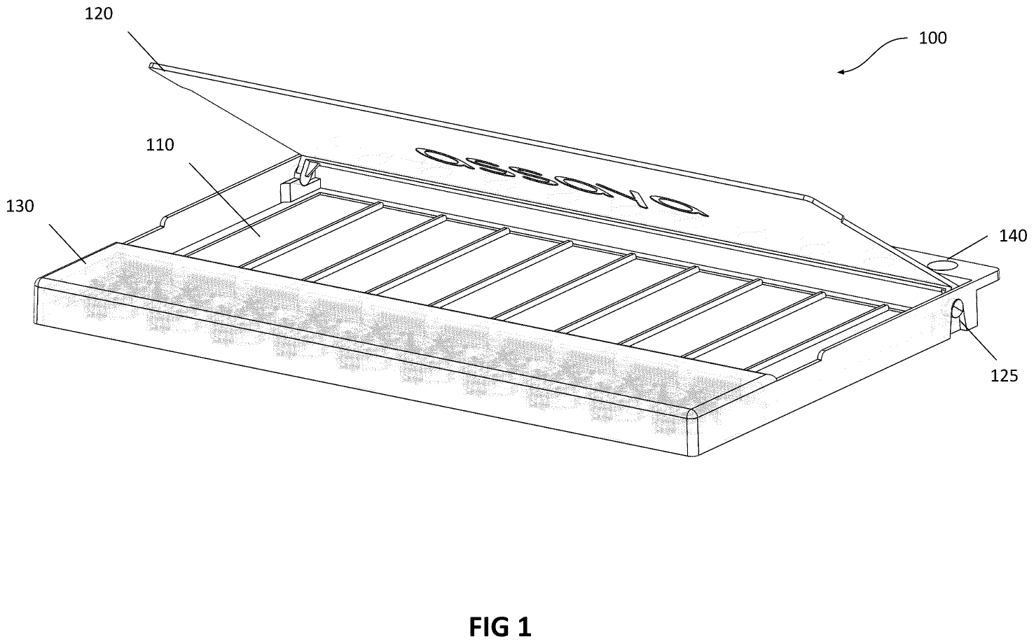

FIG. 1 depicts an orthogonal view of a timer tray 100 with an open lid according to a first embodiment of the present invention. The timer tray 100 holds a plurality of cassettes within a series of cassette holders 110. The exemplary timer tray 100 holds 10 cassettes, but one of ordinary skill in the art after reading the present disclosure would appreciate that the timer tray 100 could have a lesser or greater number of cassette holders 100. The cassettes within the cassette holders 110 are protected from contamination by a lid 120 rotatably and removable affixed within the timer tray 100 by two hinges 125. The lid 120 may have a logo or trademark, in this case Assaya™, that may be embossed or silk-screened onto the lid 120. An interface panel 130 on the surface of timer tray 100 is at least partially translucent and covers a circuit board that has an LCD display, multicolor LED light, and switch associated with each cassette holder 110. By having the interface panel 130 be a solid, continuous surface, the circuit board, and associated LCD display and switch, is protected from dirt and contamination. It also permits easier cleaning of the timer tray 100. A sample holder 140 has a plurality of respective sample receptacles for holding sample tubes containing the sample that has been placed on the sample pad of the LFA strip within the cassette in the respective cassette holder 110.

When a technician places a cassette into cassette holder 110, she depresses below on the interface panel 130, known as an initial press, which triggers a switch triggering the start of a countdown timer on the LCD display associated with the cassette holder 110. Furthermore, the multicolor LED light is turned yellow to indicate an incubation period is in progress. When countdown is complete, the multicolor LED light turns green to indicate that the cassette incubation time is complete and the cassette may be removed and the LFA strip read for results. Upon removing the cassette from cassette holder 110, the technician would again press the switch as a retrieval press indicating that the cassette has been retrieved. This would turn off the multicolor LED. If the technician fails to press the switch or remove the cassette, the multicolor LED will turn red after a preset period of time to indicate that the LFA strip results will not be accurate as the technician has waited too long to read the LFA strip. Long pressing the switch turns off the multicolor LED and resets the timer.

In addition, the switch pressing described above may be replaced by a switch, such as a pressure switch or proximity switch, mounted on the bottom of the cassette holder 110 that acts as the initial switch press when the cassette is placed in the cassette holder 110 and acts as the retrieval press when the cassette is removed from the cassette holder 110.

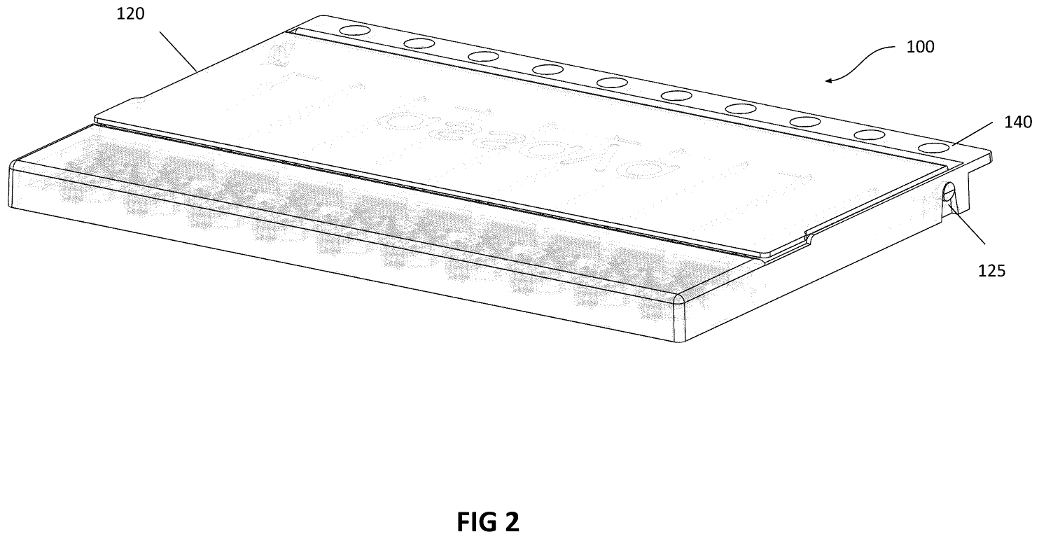

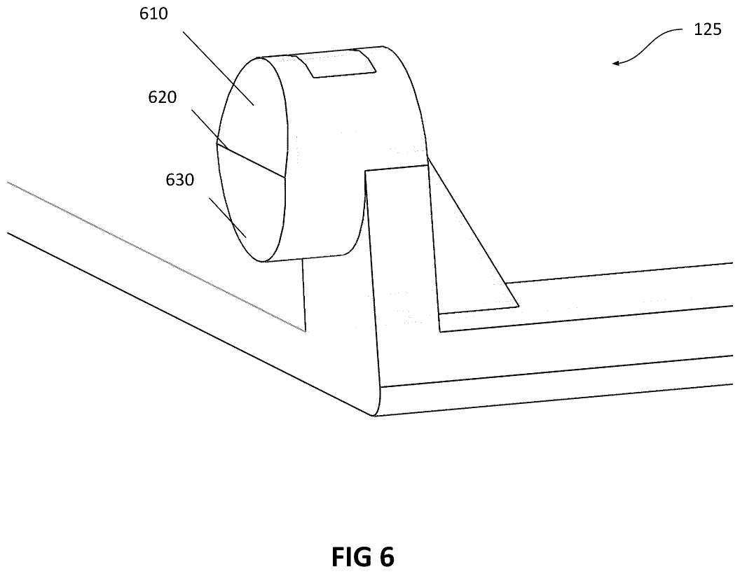

FIG. 2 depicts an orthogonal view of the timer tray 100 with the lid 120 closed according to the first embodiment of the present invention. This Figure shows the lid 120 in a closed position protecting the LFA strips in the cassette from contamination. Lid 120 is removable, as well as rotatable, and details of the hinge 125 will be shown in more detail in FIG. 6.

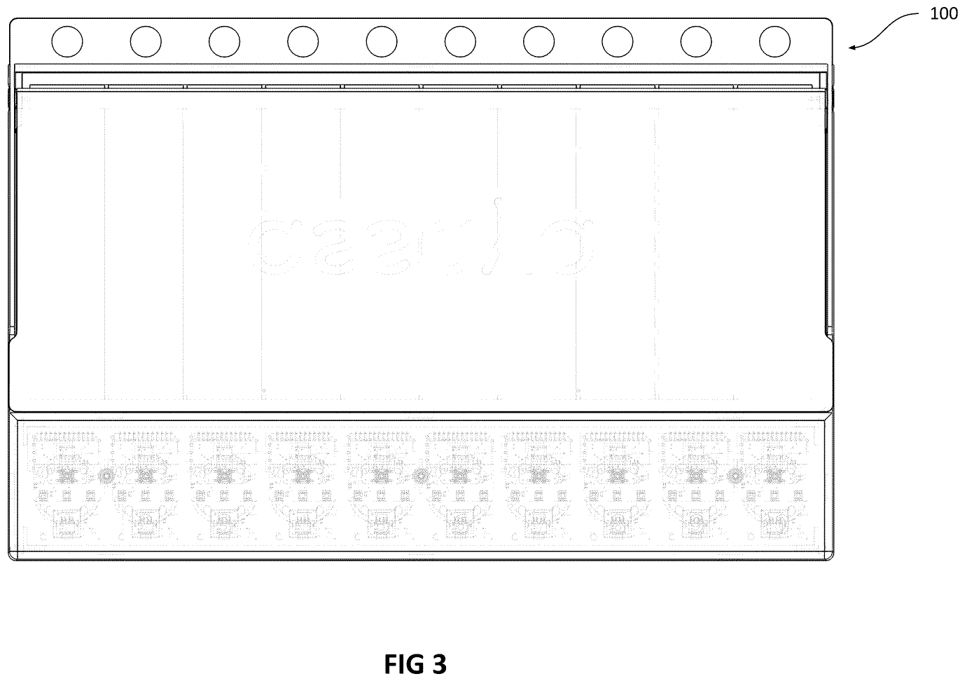

FIG. 3 depicts a top view of the timer tray 100 according to the first embodiment of the present invention. In this view, one can more clearly see a logo, in this case Assaya™, that may be embossed or silk-screened onto the lid 120.

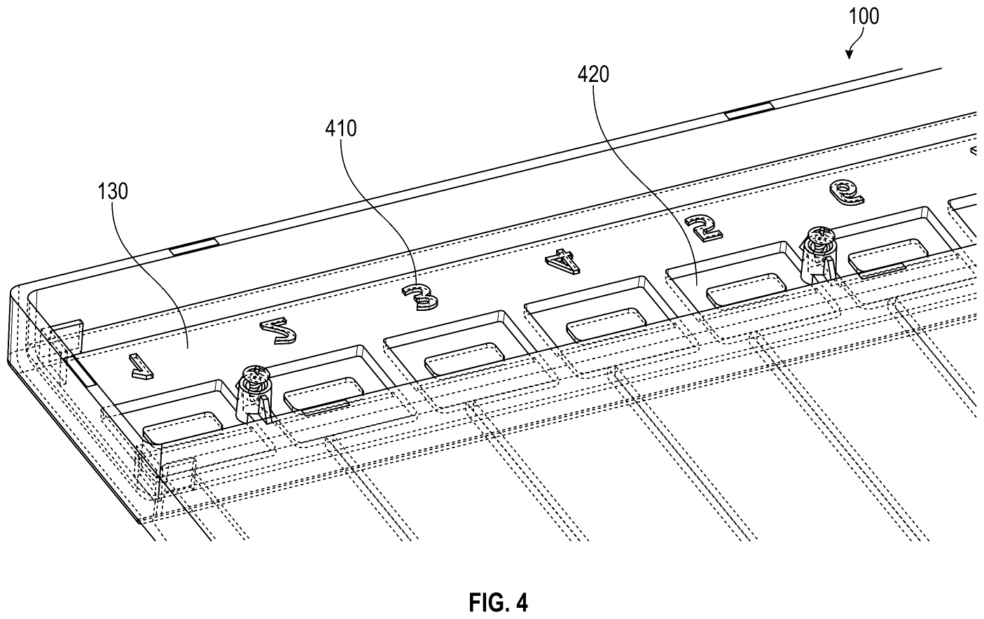

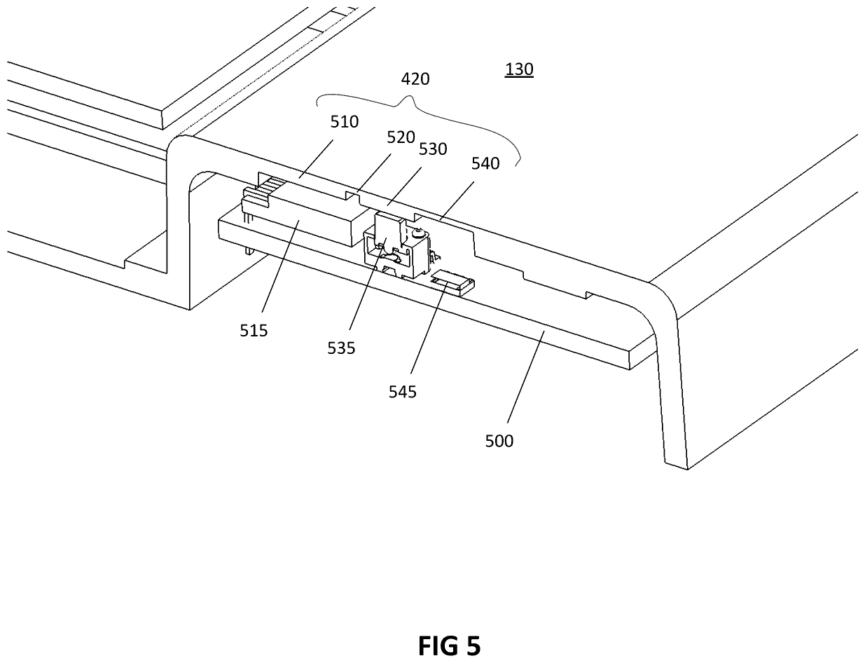

FIG. 4 depicts an orthogonal view of the underside of the timer tray 100 with the circuit board removed according to the first embodiment of the present invention. The circuit board is removed in this Figure to more clearly show the underside of the interface panel 130. In an embodiment of the invention, the top surface of the interface panel 130 is a smooth surface, with the underside having molded numbers 410 for each cassette holder placed on the underside of the interface panel 130. A plurality of respective switch and display areas 420 are molded into the interface panel 130 having various thicknesses that permit flexibility, so that when the interface panel 130 is depressed respective switches below the interface panel are activated. Furthermore, the respective switch and display areas have a thickness above the LCD display and multicolor LED, so that they may be seen through the interface panel 130. This is seen more clearly in FIG. 5 described below.

FIG. 5 depicts an orthogonal view of a cross-section of the timer tray 100 at the switch and display areas 420 below the interface panel 130 according to the first embodiment of the present invention. Below interface panel 130, lies circuit board 500 having a plurality of respective sets of LCD displays 515, switches 535, and multicolor LEDs 545, with each set associated with a respective cassette holder 110. Each respective switch and display area 420 has a thickness 510 above the LCD displays 515, thickness 530 above the switches 535, and thickness 540 above the multicolor LEDs 545 such that the LCD displays 515 and multicolor LEDs 545 are visible through the interface panel 130. Furthermore, thicknesses 520 and 540 are designed to permit sufficient flexibility in the interface panel 130 to permit switch 535 to be depressed when thickness area 530 is depressed.

FIG. 6 depicts an orthogonal view of the hinge 125 of the lid 120 of the timer tray 100 according to the first embodiment of the present invention. The hinge 120 includes a mold line 620 separating a first surface 610 and a second surface 630. The first surface 610 and second surface 630 are angles to permit easy insertion and removal of lid 120.

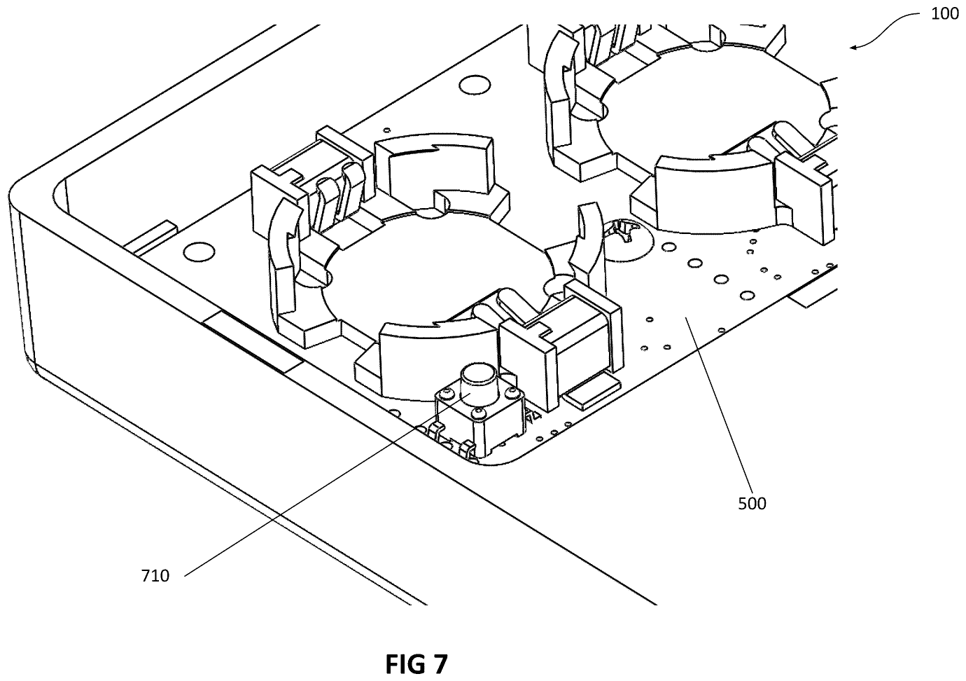

FIG. 7 depicts an orthogonal view of the underside of the circuit board 500 of the timer tray according to the first embodiment of the present invention. The circuit board 500 contains a programming switch 710. When the programming switch 710 is long depressed, the preset time of the timer tray 100 may be changed by depressing one of the switches 535 under the surface of the interface panel 130. Each of the respective switches 535 beneath the interface panel is associated with a different preset time. For example, the switch 535 below cassette holder 110 Number 1, may be associated with one minute, with Number 2 five minutes, with Number 3 15 minutes, etc. These times may be preset and stored in nonvolatile memory of a processor on circuit board 500 and may be changed by downloading new values to the processor through, for example, a USB-C interface.

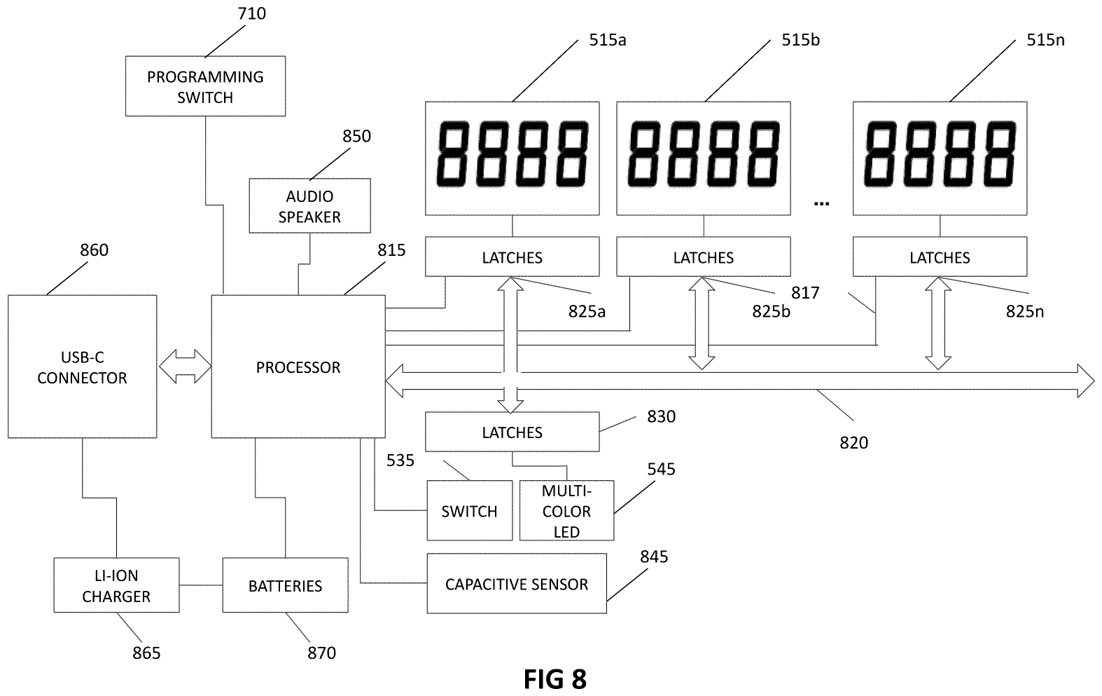

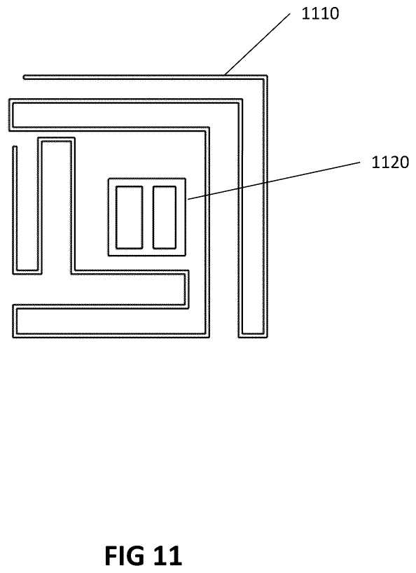

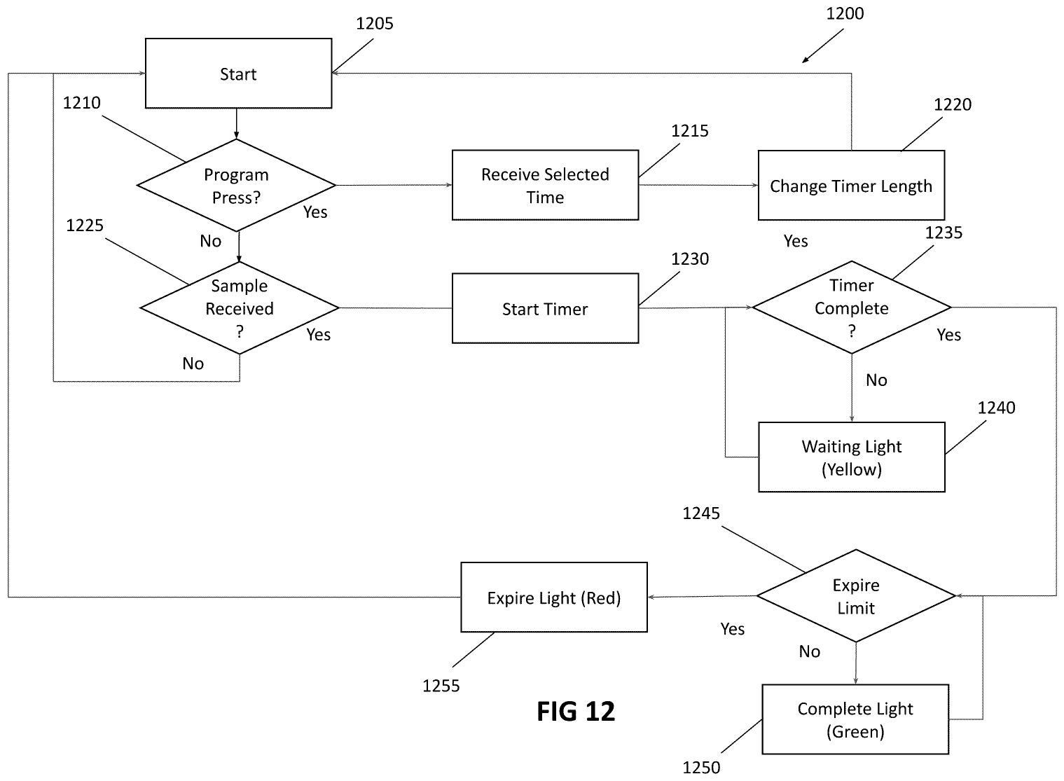

FIG. 8 depicts a block diagram of the electronic circuitry on the circuit board 500 of the timer tray 100 according to the first or a second embodiment of the present invention. The second embodiment of the present invention will be discussed in FIGS. 11 and 12 below, but uses similar circuitry as will be described in this Figure. A processor 815, such as a microcontroller, microprocessor, digital signal processor, or other timing control with internal or external memory including non-volatile memory coupled to the processor, controls the timer tray 100 and implements the timing function and operates the various displays. The microcontroller 815 is in communication with the plurality of LCD displays 515 through bus 820 in communication with a plurality of respective latches 825 in communication to respective LCD displays 515. A plurality of respective enable lines 817 runs from the processor to each respective latch. As the processor 815 outputs a time for display on an LCD display 515 it enables the LCD display's 515 respective latch 825 to hold that value for display on its respective LCD display 515. Thus, a single bus 820 may transmit data to a plurality of LCD displays 515.

Respective multicolor LED latches 830, one for each cassette holder 110, are in communication with bus 820 and multicolor LEDs 545, again one for each cassette holder 110. Through this arrangement, the processor 815 may set the color of the multi-color LED 545. Each switch 535 associated with each cassette holder 110 is in communication with the processor 815 to provide an input to the processor 815 when a cassette is placed into cassette holder 110. In an alternative embodiment, a capacitive sensor 845 or proximity sensor is in communication with processor 815 to provide an input as cassettes are inserted and removed from cassette holder 110.

An audio speaker 850 is in communication with processor 815 to provide various sounds or speech as timers are initiated, completed, or expired. Programming switch 710, discussed with respect to FIG. 7, is in communication with processor 815 in order to change timer values as previously discussed. A USB-C interface 860, or other interface is in communication with processor 815 to provide power through a charger 865 to charge one or more batteries 870 that power timer tray 100. The USB-C interface 860 is also used to change preset time values stored in non-volatile memory of processor 815. The USB-C interface 860 is also used to communicate with external computers in order to receive firmware downloads and upload statistical and other datas collected by the timer tray 100. The USB-C interface 860 via an external computer uploads data to a central data center or anywhere in the Internet cloud with proper security permissions in place. While the USB-C interface 860 is the primary method of uploading to the cloud, those of ordinary skill in the art after reading this specification will appreciate that direct network connections, such as WiFi, Bluetooth, Ethernet, or Mobile interfaces, may also be used for data upload and timer preset and firmware downloads.

In addition to controlling timing, processor 815 in conjunction with its internal or external non-volatile memory stores data regarding the timing function. Statistics regarding the timer tray 100 are stored, including, for example, the number of times that each of the n timers is activated, number of times the timers reached an expiration, time, and date is stored in the non-volatile memory. The timer tray 100 may be equipped with temperature and/or humidity sensors, and, if so, humidity and temperature data is also stored in non-volatile memory. This data is acceptable from processor 815 through USB-C interface 860.

Processor 815 also senses battery 870 voltage and when the battery 870 voltage is below a lower threshold where there is a risk of a shutdown for insufficient energy. When the battery 870 state gets below the threshold amount, all timer states in process have the times stored in the non-volatile memory, such that upon restart following a change of battery 870 the timer states are restored so that timers are not lost upon battery 870 change.

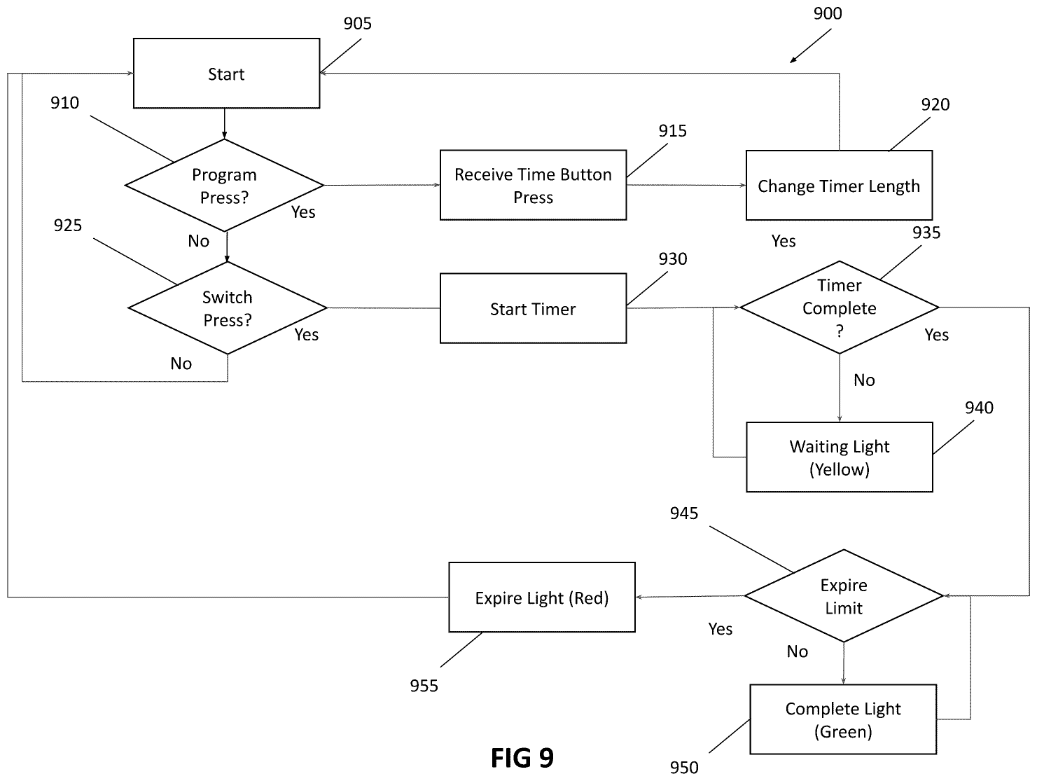

FIG. 9 depicts a flow chart 900 of the operation of the circuit board 500 of the timer tray 100 according to the first embodiment of the present invention. Upon power-up, the tier board enters into a start state (block 905). When the programming switch 710 is pressed (block 910), the processor 815 waits to receive a press of one of the switches 535 (block 915). When pressed, the processor 815 changes the length of the incubation timer 920 based upon which switch 535 has been activated (block 920). Flow then returns to start block 905.

If the program switch 710 has not been pressed (block 910), a check is made to determine if a switch 535 press has occurred (block 925). If so, the timer associated with that switch 535 is activated (block 930). If the timer is complete (block 935) and the expiration limit has not been exceeded (block 945), the respective multicolor LED 535 is lit green (block 950). If the timer is not yet complete (block 935), the multicolor LED 545 is lit yellow to indicate a waiting for completion of incubation state (block 940). If the timer is complete (block 935) and the expiration limit has been exceeded (block 945), the multicolor LED 955) is lit red to indicate expiration of the time to get accurate results from the LFA strip in the carrier. This method 900 is simultaneously running for each of the respective circuitry associated with its respective carrier.

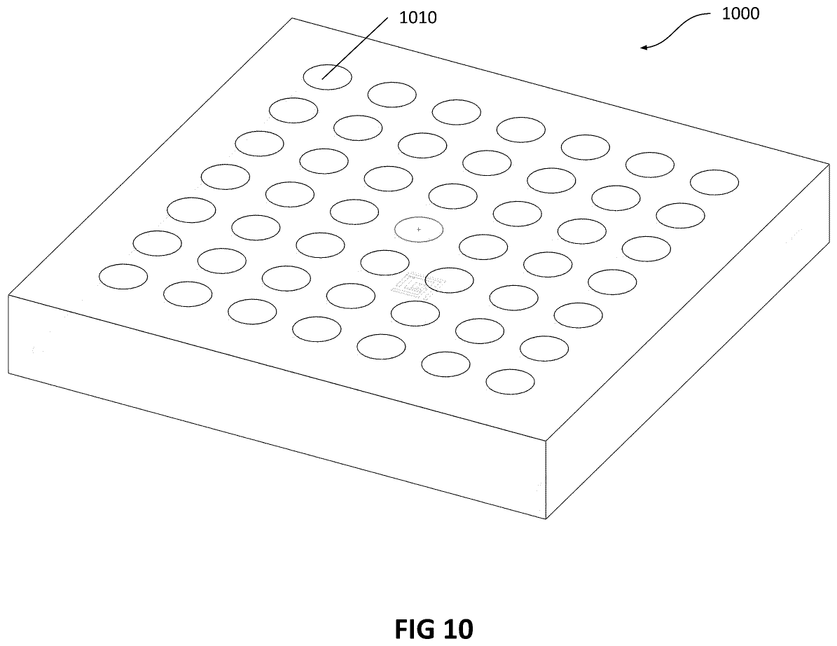

FIG. 10 depicts an orthogonal view of a timer tray 1000 in accordance with a second embodiment of the present invention. The timer tray 1000 has a plurality of receptacles 1010 for holding samples awaiting incubation completion. Each receptacle has a sensor (such as capacitive sensor 845) at the bottom of the receptacle, along with a multicolor LED 1120 (shown in FIG. 11). The sensor 845 detects the presence or absence of a sample, and replaces the need for a manual switch such as switch 535. Upon receipt of a sample, sensor 845 communicates its presence to the processor 815 to initiate the timer, and upon removal of the sample the sensor 845 communicates its absence to the processor 815 to stop and reset the associated timer. Thus, manual button presses are not needed as they may be in the first embodiment. A programming switch 710 (not shown in this Figure) is also provided and operates as described with respect to the first embodiment. The multicolor LED 1120 will shine through the sample in order for a technician to see the state of the incubation (waiting/yellow, ready/green, or expired/red).

FIG. 11 depicts the capacitance switch 845 and multicolor LED 1120 placed at the bottom of each receptacle in accordance with the second embodiment of the present invention. Each operates as described with respect to FIG. 8.

FIG. 12 depicts a flow chart 1200 of the operation of the circuit board 500 of the timer tray 1000 according to the second embodiment of the present invention. Upon power-up, the tier board enters into a start state (block 1205). When the programming switch 710 is pressed (block 1210), the processor 815 waits to receive an indication of the presence of a sample at one of the sensors 845 (block 1215). When a sensor is activated, the processor 815 changes the length of the incubation timer based upon which sensor 845 has been activated (block 1220). Flow then returns to start block 1205.

In an alternative embodiment, in place of waiting for placements of a sample to receive instructions on incubation timer length, a long press of programming switch 710 may place timer tray 1000 in programming mode, with repeated short presses of programming switch 1000 resulting in sequential lighting of multicolor LED 545 through the range of receptacles with each receptacle representing a preset time. Once short pressing ceases, the incubation time is set based on the receptacle selected.

If the program switch 710 has not been pressed (block 1210), a check is made to determine if a sample press has been received (block 1225). If so, the timer associated with that receptacle is activated (block 1230). If the timer is complete (block 1235) and the expiration limit has not been exceeded (block 1245), the respective multicolor LED 1120 is lit green (block 1250). If the timer is not yet complete (block 1235), the multicolor LED 1120 is lit yellow to indicate a waiting for completion of incubation state (block 1240). If the timer is complete (block 1235) and the expiration limit has been exceeded (block 1245), the multicolor LED 1120) is lit red to indicate expiration of the time to get accurate results from the sample in the receptacle. This method 1200 is simultaneously running for each of the respective circuitry associated with its respective receptacle.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A device, comprising:
a processor;
memory coupled to the processor;
a display coupled to the processor;
a switch coupled to the processor; and
a cassette holder for holding a cassette associated with the display and the switch,
wherein when the processor receives an input indicating activation of the switch, a timer is activated for a predetermined timer length and time is displayed on the display.

2. The device of claim 1 further comprising a multicolor light for indicating the state of the timer.

3. The device of claim 1, wherein the timer is a countdown timer.

4. The device of claim 1, wherein the timer is a countup timer.

5. The device of claim 2, wherein the multicolor light indicates a waiting state when the timer is active and not yet expired.

6. The device of claim 2, wherein the multicolor light indicates a ready state when the timer is complete and not yet expired.

7. The device of claim 2, wherein the multicolor light indicates an expired state when the timer is complete and expired.

8. The device of claim 1, wherein the switch is activated upon placing the cassette into the cassette holder.

9. The device of claim 8, wherein the switch is deactivated upon removing the cassette from the cassette holder.

10. The device of claim 1, further comprising a programming switch in communication with the processor, wherein activation of the programming switch puts the processor in programming mode for changing the preset time length.

11. The device of claim 10, wherein activation of the switch changes the preset time.

12. A device, comprising:
a processor;
memory coupled to the processor;
a sensor coupled to the processor; and
a receptacle for holding a sample associated with the switch,
wherein when the processor receives an input indicating activation of the sensor, a timer is activated for a predetermined timer length.

13. The device of claim 12 further comprising a multicolor light for indicating the state of the timer.

14. The device of claim 12, wherein the timer is a countdown timer.

15. The device of claim 12, wherein the timer is a countup timer.

16. The device of claim 13, wherein the multicolor light indicates a waiting state when the timer is active and not yet expired.

17. The device of claim 13, wherein the multicolor light indicates a ready state when the timer is complete and not yet expired.

18. The device of claim 13, wherein the multicolor light indicates an expired state when the timer is complete and expired.

19. The device of claim 1, further comprising a programming switch in communication with the processor, wherein activation of the programming switch puts the processor in programming mode for changing the preset time length.

20. The device of claim 10, wherein activation of the sensor changes the preset time
